(19) United States
(12) Patent Application Publication
(10) Pub. No.: US 2006/0234937 A1
(43) Pub. Date: Oct. 19, 2006

(54) Modulation of angiogenesis

(75) Inventors: Terence Howard Rabbitts, Cambridge (GB); Yoshihiro Yamada, Kyoto (JP)

Correspondence Address: WILMER CUTLER PICKERING HALE AND DORR LLP, 1875 PENNSYLVANIA AVE., NW, WASHINGTON, DC, 20004, US

(73) Assignee: Medical Research Council, London (GB)

(21) Appl. No.: 11/447,882

(22) Filed: Jun. 7, 2006

Related U.S. Application Data
(63) Continuation of application No. 10/954,175, filed on Oct. 1, 2004, now abandoned.
Application No. 10/954,175 is a division of application No. 10/900,464, filed on Jul. 28, 2004, now abandoned.
Application No. 10/900,464 is a division of application No. 10/110,999, filed on Apr. 19, 2002, now abandoned.

(30) Foreign Application Priority Data
Oct. 20, 1999 (EP) 9924858.5

(51) Int. Cl. A61K 38/17

(52) U.S. Cl. 514/12

(57) ABSTRACT

Substances are provided that are capable of modulating angiogenesis mediated by Lmo2 or a functionally related polypeptide, which substance binds to Lmo2 and/or a functionally related polypeptide, or alters the expression of Lmo2 or a functionally related polypeptide in a cell. Assay methods are provided for identifying such substances.

### FIELD OF THE INVENTION

The present invention relates to assay methods for identifying substances capable of modulating angiogenesis mediated by Lmo2 or a functionally related polypeptide. The present invention also relates to substances capable of modulating angiogenesis by their effect on Lmo2 and their use in treating pathological conditions characterised by angiogenesis.

### BACKGROUND TO THE INVENTION

The development of a vascular supply is essential for the growth, maturation, and maintenance of normal tissues. It is also required for wound healing and the growth of solid tumours and is involved in a variety of other pathological conditions. Current concepts of angiogenesis suggest that cells secrete angiogenic factors which induce endothelial cell migration, proliferation, and capillary formation.

The major development of the vascular supply occurs during embryonic development, at ovulation during formation of the corpus luteum, and during wound and fracture healing. However, many pathological disease states are characterised by augmented angiogenesis including tumour growth, diabetic retinopathy, neovascular glaucoma, psoriasis, and rheumatoid arthritis among other conditions. During these processes normally quiescent endothelial cells which line the blood vessels sprout from sites along the vessel, degrade extracellular matrix barriers, proliferate, and migrate to form new vessels. These processes are believed to be induced by factors secreted by the tissues to be vascularised and are often referred to as angiogenic factors. Angiogenic factors are secreted from surrounding tissue during the process which directs the endothelial cells to degrade stromal collagens, undergo directed migration (chemotaxis), proliferate, and reorganise into capillaries.

In the normal course of the growth of a mammalian embryo (embryogenesis), development of a vascular system is essential for embryos to grow after they reach a certain size. During embryogenesis, the vascular system is constructed by two distinct processes. The first is vasculogenesis, in which haemangioblasts, putative common precursors, are specified from mesoderm to form a primary capillary network. The mature vascular structures are formed by a second process called angiogenesis, which is remodelling of the existing capillary network into mature blood vessels.

The relationship between endothelial cells and blood cell specification suggests the presence of a common precursor. Primitive haematopoiesis begins around embryonic day E7.5 in yolk sac blood islands whilst definitive haematopoiesis initiates in the aorta-gonad-mesonephoros (AGM) region around E10.5. Moreover, blood cells are derived from endothelial cells of large arteries such as dorsal aorta, umbilical artery and vitelline artery in human.

Transcription factors involved in these processes are important in control of both endothelial and haematopoietic cell fate. The LIM-domain gene, LMO2, is activated by chromosomal translocations in leukaemia (Boehm et al., 1988; Boehm et al., 1991) and has an essential role in the initiation of haematopoiesis in mouse embryogenesis (Warren et al., 1994; Yamada et al., 1998).

Studies into the regulation of embryonic angiogenesis may provide important information to assist in the design of strategies for modulating tumour angiogenesis and tumour progression.

### SUMMARY OF THE INVENTION

The role of Lmo2 in the formation of the vascular system in mouse chimaeras has now been assessed by following the fate of Lmo2-null ES cells. Lmo2 was found to be expressed in embryonic endothelial cells and vasculogenesis proceeded normally in the absence of Lmo2.

However, surprisingly, Lmo2-null ES cells are unable to contribute to endothelial cells of mature blood vessels after about E11. These results show that Lmo2 is necessary for angiogenic remodelling of the existing capillary network into mature vasculature, which is a prerequisite for definitive haematopoiesis.

Moreover, we have shown that Lmo2−/− tumour cells are unable to contribute to tumour vascularisation in adult mammals.

Consequently, regulation of Lmo2 activity in tissues may provide an effective strategy for modulating angiogenesis, for example inhibiting angiogenesis in solid tumours or promoting angiogenesis in wound tissue or ischaemic tissue.

Accordingly, the present invention provides a method for modulating angiogenesis in a mammal, comprising modulating the expression and/or function of Lmo2 or polypeptide functionally related thereto.

It is known that Lmo2 is involved in the formation of a multiprotein DNA binding complex, including various partners such as Tal1, E47, GATA1 and Lbd1/NL1. Thus, the present invention relates to the modulation of Lmo2 activity, or that of any polypeptide which is involved in Lmo2 function. Preferably, the “functionally related polypeptide” is a binding partner of Lmo2. Preferably, the binding partner is Tal1.

Modulation of angiogenesis, as set out above, may comprise upregulation or downregulation thereof. It has been found that inhibition of Lmo2 expression effectively inhibits angiogenesis in mature mammals and in tissues thereof, particularly tumour tissues.

In a preferred embodiment, the invention provides a method for inhibiting the contribution of a cell to a process of angiogenesis in a mammal, comprising inhibiting the expression of Lmo2 in said cell. According to the invention, by inhibiting Lmo2 expression, the involvement of a cell in angiogenesis or its contribution thereto by involvement of the cell or progeny thereof in the angiogenesis process may be inhibited.

The invention accordingly provides the use of Lmo2 as a target for drug development. Regulators of Lmo2 are potent modulators of angiogenesis and can be identified using Lmo2-modulation assays.

In a further aspect, the invention provides a method for identifying a substance capable of modulating angiogenesis mediated by Lmo2, which method comprises contacting a cell which expresses Lmo2 or a functionally related polypeptide with a candidate substance and determining whether the activity of Lmo2 or the functionally related polypeptide is altered.

In a preferred embodiment the present invention provides a method for identifying a substance capable of inhibiting angiogenesis, which method comprises contacting a cell which expresses Lmo2 or a functionally related polypeptide with a candidate substance and determining whether expression of Lmo2 or the functionally related polypeptide is inhibited.

In another preferred embodiment the present invention provides a method for identifying a substance capable of promoting angiogenesis, which method comprises contacting a cell which expresses Lmo2 or a functionally related polypeptide with a candidate substance and determining whether expression of Lmo2 or the functionally related polypeptide is upregulated.

The present invention also provides a method for identifying a substance capable of modulating angiogenesis, which method comprises contacting an Lmo2 polypeptide or functionally related polypeptide with a candidate substance and determining whether said substance binds to the Lmo2 polypeptide or functionally related polypeptide.

In a preferred embodiment the present invention provides a method for identifying a substance capable of inhibiting angiogenesis, which method comprises contacting a cell which expresses Lmo2 or a functionally related polypeptide with a candidate substance and determining whether said substance binds to the Lmo2 polypeptide or functionally related polypeptide.

In another preferred embodiment the present invention provides a method for identifying a substance capable of promoting angiogenesis, which method comprises contacting a cell which expresses Lmo2 or a functionally related polypeptide with a candidate substance and determining whether said substance binds to the Lmo2 polypeptide or functionally related polypeptide.

The present invention also provides a method for identifying a substance capable of modulating angiogenesis mediated by Lmo2 which method comprises:

(i) providing an Lmo2 polypeptide;

(ii) providing an Lmo2 binding partner;

(iii) incubating the Lmo2 polypeptide and binding partner in the presence and absence of a candidate substance under suitable conditions; and

(iv) determining whether the candidate substance affects the interaction between Lmo2 and the binding partner.

Preferably the above methods of the invention further comprise administering a substance identified by the above methods to a mammal and determining whether angiogenesis is modulated, for example inhibited or promoted.

The results presented herein indicate that modulation of Lmo2 function, for example either by altering Lmo2 expression or by interfering with Lmo2 biological function, may be used to modulate angiogenesis in vivo, for example to inhibit or stimulate angiogenesis.

Accordingly the present invention provides a substance capable of modulating angiogenesis mediated by Lmo2 or a binding partner thereof. Preferably said modulation is inhibition or promotion of angiogenesis.

Preferably said substance is capable of altering expression of Lmo2 or a functionally related polypeptide or modulating a biological function of Lmo2 or a functionally related polypeptide. More preferably said substance is identified by above methods of the invention.

The present invention further provides a substance of the invention for use in modulating angiogenesis. The present invention also provides a method of modulating angiogenesis in a mammal which method comprises modulating expression and/or function of Lmo2 or a functionally related polypeptide.

### DESCRIPTION OF THE FIGURES

FIG. 1 Structure of Lmo2 Targeted Alleles in ES Cells Used to Make Chimaeric Mice

Heterozygous and homozygous null mutations were generated in ES cells to give lmo2+/− (KZ26+/−) and lmo2−/− (KZ26−/−).

A. The null allele in lmo2−/− was made using neomycin selection and caused a fusion of the bacterial lacZ gene with the second exon of lmo2; this results in a β-galactosidase fusion protein comprising a short stretch of lmo2 encoded amino acids and a whole βgalactosidase protein.

B. Lmo2+/− ES cells (clone KZ26+/−) were used for targeting the second Lmo2 allele by insertion of the hygromycin resistance marker. This produced the Lmo2−/− cells (KZ26−/−).

FIG. 2. Lmo2-lacZ Fusion Gene Knock-In by Homologous Recombination

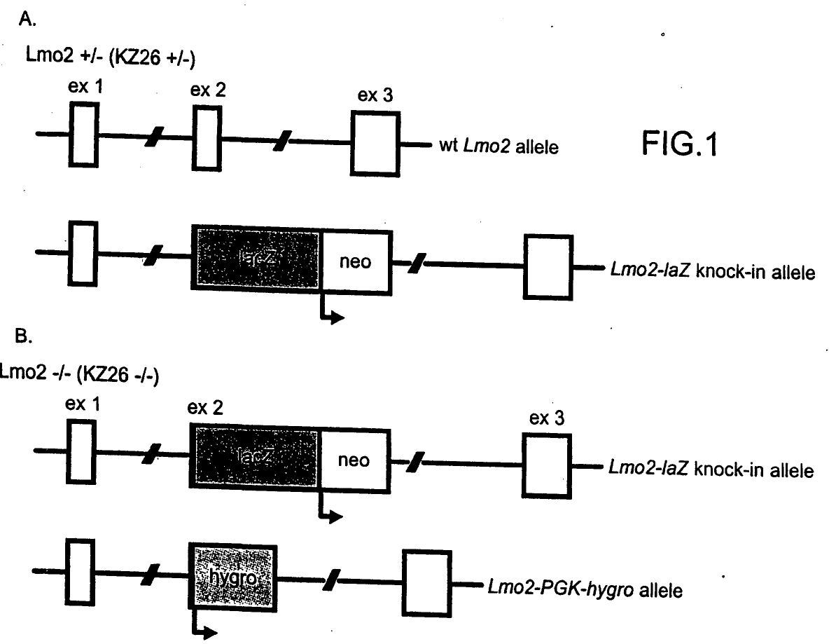

Two constructs were used to create the Lmo2 targeted ES cells used in this study. CCB ES cells were transfected with pKO5-lacZ-neo (A) and targeted events detected by filter hybridisation. Several targeted clones were initially analysed and one (designated KZ26) was chosen based on its ability to consistently yield high levels of chimaerism in mice after injection into blastocysts. The KZ26 clone +/−was used for a second transfection with pKO5hygro(tk) and three clones (1, 16 and 64) were studied in which the second allele of Lmo2 had been targeted to yield KZ26−/− (Lmo2−/−) ES cells.

Construction of the Lmo2-lacZ fusion gene targeting vector was done by cloning of 4.5 kb blunt ended SfiI fragment of SfiI-lacZ-MC1neopA (Dear et al., 1995) into blunt-ended BamHI site of gene targeting vector pKO5(tk) (Warren et al., 1994). In the resulting clone, KO5-lacZ-neo, the 24^{th} codon of Lmo2 (exon2) was linked to 2^{nd} codon of lacZ by 12 bp linker sequence. The hygromycin targeting vector (pKO5hygro(tk) has been described (Warren et al., 1994). The location of the probes used to assess gene targeting is indicated on the map of pKO5-lacZ-neo and have been previously described (Warren et al., 1994) The targeting of the pKO5-lacZ-neo into Lmo2 yields a 6 Kb SacI fragment with probe A (compared with a 9 Kb germ-line band) and a 9.5 Kb BamHI band with probe B (compared with a 12 Kb germ-line band) (Warren et al., 1994). Probe C is a neo probe used to verify a single insertion of the targeting vector. Targeting of pKO5hygro(tk) into Lmo2 yields a 10.8 Kb SacI band with probe A and 13.8 Kb BamHI band with probe B. S=SacI; B=BamHI

Detection of homologous recombination in ES clone DNA by Southern filter hybridisation with probe A (3′ flanking), probe B (5′ flanking) and probe C (internal). Hybridisation of representative +/+ (wild-type) and +/− (neo-targeted) clones are shown. Wt=wild type hybridisation band

C. Identification of three independent double targeted Lmo2 −/− clones (clone 1, clone16 and clone64) by filter hybridisation with probe A. The integrity of these second targeted alleles was verified using probe B and an internal hygromycin probe (data not shown).

FIG. 3. Whole Mount Beta-Galactosidase Staining of Lmo2+/− Heterozygous Embryos

The targeted ES clone KZ26 (with one null Lmo2 allele) was injected into blastocysts, chimaeric mice obtained and germ-line transmission of the Lmo2 null allele was obtained. Heterozygous KZ26 mice were crossed with C57/B16 mice and embryos obtained at E10.5 and E12.5. These embryos were stained with X-gal as a substrate for beta-galactosidase activity. Blue staining denotes areas of beta-galactosidase due to Lmo2-lacZ gene expression.

A. E10.5 embryo. X-gal staining was seen on major blood vessel walls and capillaries of whole body.

B. E12.5 embryo. In addition to beta-galactosidase staining of vasculature, prominent staining was found in the limb buds and the tip of tail.

C. Histological section of an E12.5 embryo stained for beta-galactosidase and counter-stained with eosin. Blood vessel endothelial cells can be observed in a background of eosin stained tissue.

D. Histological section of the limb bud of an E12.5 embryo stained for beta-galactosidase and counter stained with eosin. The region beneath the apical ectodermal ridge of a limb bud is beta-galactosidase positive.

FIG. 4. Comparison of Whole Mount X-Gal Staining of KZ26+/−and KZ26 −/−Chimaeric Embryos.

ES cells with one (KZ26 +/−) or two (KZ26 −/−) Lmo2 null alleles were injected into C57/B16 blastocysts and transferred to recipient females. At the indicated embryonic days, embryos were dissected and whole mount stained with X-gal for beta-galactosidase activity due to Lmo2-lacZ gene expression.

A. E11.5 KZ26 +/− chimaeric embryo with staining pattern similar to that seen in heterozygous KZ26 mice.

B. E11.5 KZ26 −/− chimaeric embryo derived from KZ26 −/− clone 1. In this embryo, ES cell contribution (blue) can be seen in hippocampus and limb buds and very few endothelial cells stained blue (i.e. those of ES cell origin).

E12.5 KZ26+/− chimaeric embryo. Like the E11.5 KZ26 +/− embryo, the staining pattern was very similar to that seen in heterozygous KZ26 mice.

E and F. E12.5 KZ26 −/− chimaeric embryos of three independent −/− clones.

D. An embryo derived from injection of KZ26 −/− clone 1, E. an embryo from clone 16 and F. an embryo from clone 64. In KZ26 −/− chimaeric embryos post-E11.5, there was no contribution of −/− ES cells in endothelial cells of major vessels whilst there is maintained expression in the hippocampus, the limb bud and tail. This selective loss of ES contribution in blood vessel endothelium suggests an essential role of Lmo2 protein in the maturation of vascular network (angiogenesis).

FIG. 5. Growth Retardation and Disorganisation of the Vascular System in E10.5 Lmo2 −/− Chimaeric Embryos

Chimaeric embryos were generated by injection of KZ26 +/− ES cells (A) or KZ26 −/− ES cells clone 1 (B-J) into blastocysts, obtaining embryos at E10.5 and whole mount X-gal staining.

The embryos shown in B-J are litter-mates. The difference in the size and X-gal staining indicates an inverse relationship between size and level of ES cell contribution, as the higher chimaeras (i.e. those with higher levels of beta-galactosidase staining) are growth retarded.

A. E10.5 KZ26 +/− chimaeric embryo. This photograph is of a representative embryo and there was no marked size difference among the litter mates.

B to J. A series of whole mount X-gal staining of KZ26 −/− chimaeric embryos.

From A to H, magnification rate is the same.

The enlarged figure (approximately 1.7×) of embryo D to show the major disorganisation in vasculature of this high chimaeric −/− embryo. No mature vessels were found in comparison with KZ26+/− (A).

K. Histological section of −/− chimaeric embryo D stained with X-gal and counter stained with eosin.

FIG. 6. Lmo2 Expression in Normal Adult Vasculature and Tumour Vessels in Lmo2-lacZ Gene Fusion Knock-In Mice.

Tissues were dissected from normal or tumour-bearing Lmo2-lacZ gene fusion knock-in mice (KZ26; Lmo2+/−) (9 weeks-15 weeks old) for whole mount X-gal staining. Organs from wild type mice were stained with the same method to exclude endogenous activity of p-galactosidase (data not shown). A. Whole mount of Lmo2+/− mouse brain; B. Whole mount of Lmo2 +/− mouse kidney (note Xgal staining in glomeruli in the cortex); C. Whole mount of Lmo2 +/− mouse liver; D. Whole mount of a subcutaneous tumour produced by Lewis lung cell carcinoma cell implantation into C57B16 mouse with Lmo2 +/− genotype; E. Whole mount of an abdominal lymph node tumour (T cell lymphoma) which developed in Lmo2 +/; p53 −/− mouse; F. Histological section of an X-gal stained tumour sample from an abdominal lymph node shown in panel E, counter-stained with nuclear fast red. Tumour vessel endothelium is stained blue.

c=cortex; m=medulla; en=endothelium

FIG. 7. Augmented Lmo2 Expression in Thymoma Blood Vessel Endothelium.

Thymus was taken from a Lmo2 +/− mouse (A, D) and thymomas from Lmo2 +/− crossed with p53 knock-out mice (B, E) (genotype Lmo2 +/−; p53 −/−) and CD2-rbtn2/lmo2 transgene (C, F) (genotype Lmo2+/−; CD2-rbtn2+). Tissues were whole mount stained with Xgal (A-C) or histologically sectioned and counterstained with nuclear fast red (D-F). All tumours which developed in control mice with Lmo2 +/+; p53 −/− genotype and Lmo2 +/+; CD2rbtn2 transgene-positive genotype were examined for endogenous lacZ activity and none showed any staining.

FIG. 8. The Development of ES-Cell Derived Vascular Trees in Subcutaneous Teratocarcinomas

Subcutaneous teratocarcinomas were produced in nude mice by implantation of heterozygous Lmo2 +/− or homozygous null Lmo2 −/− ES cells. 2×10⁶ of either Lmo2 +/− or Lmo2 −/− ES cells were implanted into both flanks of a cohort of nude mice. Subcutaneous tumours were dissected from the recipient mice at 2, 3, 3.5, 4 and 8 weeks after implantation, followed by whole mount X-gal staining and photography. Vessels of ES-cell origin (i.e. Lmo2-targeted) are distinguishable from those of host (nude mice) origin by their blue colour after X-gal staining due to the knock-in fusion of lacZ into the Lmo2 gene³. Tumour whole mount staining is shown from biopsies taken at 2 weeks (2W), 3 weeks (3W), 3.5 weeks (3.5W) and 4 weeks (4W). There is no obvious ES-derived vascular development 2 weeks after implantation in Lmo2 +/− ES cell tumours (A) or Lmo2 −/− ES cell tumours (B) as only clusters of blue cells (Lmo2 expressing cell) clusters were observed. However, at 3 weeks we noted the beginning of vascular tree development in Lmo2 +/− (C) but not −/− (D); this is very clear at 3.5 week Lmo2 +/− tumours (E) but there was no evidence at all in Lmo2 −/− tumours (F). At 4 weeks, well developed vascular trees were seen in Lmo2 +/− tumours (G), whilst there was still no sign of vascular development from Lmo2 −/− ES-derived endothelium at 4 weeks (H). In total, we analysed 19 tumours induced from each ES cell genotype (see table 1).

vt=vascular endothelium

### DETAILED DESCRIPTION OF THE INVENTION

Although in general the techniques mentioned herein are well known in the art, reference may be made in particular to Sambrook et al., Molecular Cloning, A Laboratory Manual (1989) nd Ausubel et al., Short Protocols in Molecular Biology (1999) 4^{th} Ed, John Wiley & Sons, Inc.

A. Lmo2 and Functionally Related Proteins

Lmo2

Lmo2 as used herein refers to H. sapiens Lmo2 or homologues thereof, in particular homologues thereof having an analogous biological function (i.e. promote angiogenesis). H. sapiens Lmo2 has been cloned by Royer-Pokora et al. (1991) and its amino acid and nucleotide sequence is available in the Genbank, accession no. NM_{—}005574 (version gi: 5031878). Lmo2 is also referred in the art as TTG-2, rbtn2 and rhom-2. The sequence of rhom-2 is described in Boehm et al., 1991 (Genbank accession no. M64357). See also Foroni et al., 1992.

It will be understood that Lmo2 amino acid sequences for use in the invention are not limited to the H. sapiens Lmo2 as given in Royer-Pokora et al. (1991) or sequences obtained therefrom protein but also include homologous sequences obtained from any source, for example related viral/bacterial proteins, cellular homologues and synthetic peptides, as well as variants or derivatives thereof. By way of example, several homologous mouse sequences are found in the Genbank database (such as M64360 and M64359). Lmo2 may thus be sourced from other mammalian species, such as mouse and particularly agriculturally relevant species such as cow and sheep. A high degree of sequence identity exists between mammalian homologues of Lmo2.

Thus, the present invention encompasses the use of variants, homologues or derivatives of H sapiens Lmo2 amino acid sequences, as well as variants, homologues or derivatives of the nucleotide sequences coding for the amino acid sequences.

In the context of the present invention, a homologous sequence is taken to include an amino acid sequence which is at least 60, 70, 80 or 90% identical, preferably at least 95 or 98% identical at the amino acid level over at least 30, 40 or 50 preferably 100 or 150 amino acids with the H. sapiens Lmo2 sequence (NM 005574). In particular, homology should typically be considered with respect to those regions of the sequence known to be essential for mediating angiogenesis rather than non-essential neighbouring sequences. Particularly preferred regions for homology comparison purposes are either or both of the LIM domains (approximately amino acids 28 to 82 and amino acids 92 to 149—the consensus for this type of domain is CX₂C X₁₅₋₁₇ HX₂CX₂CX₂C X₁₅₋₁₇ CX₂ (C/D/H), where X is any amino acid). Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

In the assays described below, such as in vitro binding assays, it may not be necessary to use the full length polypeptide: fragments of the full length sequence may also be used. Such fragments comprise at least 20, 30, 40 or 50 amino acids, more preferably at least 100 or 150 amino acids of the full length sequence and preferably encompass the first and/or second LIM domain (see above)

The terms “variant” or “derivative” in relation to the amino acid sequences of the present invention includes any substitution of, variation of, modification of, replacement of, deletion of or addition of one (or more) amino acids from or to the sequence providing the resultant amino acid sequence has biological activity, such as activity in mediating angiogenesis, preferably having at least the same activity as the wild type H. sapiens Lmo2 protein.

Lmo2 sequences may be modified for use in the present invention. Typically, modifications are made that maintain the activity of the sequence. Amino acid substitutions may be made, for example from 1, 2 or 3 to 10, 20 or 30 substitutions-provided that the modified sequence retains biological activity. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Isolated proteins for use in the invention are typically made by recombinant means. However they may also be made by synthetic means using techniques well known to skilled persons such as solid phase synthesis. Alternatively, they may be purified from cells that naturally express Lmo2 protein.

Proteins for use in the invention may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the biological activity of the protein of interest sequence.

Proteins for use in certain aspects of the invention, such as in vitro binding assays, may be in a substantially isolated form. It will be understood that the protein may be mixed with carriers or diluents which will not interfere with the intended purpose of the protein and still be regarded as substantially isolated. A protein for use in certain aspects of the invention, such as in vitro binding assays, may also be in a substantially purified form, in which case it will generally comprise the protein in a preparation in which more than 90%, e.g. 95%, 98% or 99% of the protein in the preparation is a protein of the invention.

Polynucleotides for use in the invention comprise nucleic acid sequences encoding Lmo2 polypeptides as described above. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides to reflect the codon usage of any particular host organism in which the polypeptides of the invention are to be expressed.

Polynucleotides for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence for use in the present invention include any substitution of, variation of, modification of, replacement of, deletion of or addition of one (or more) nucleic acid from or to the sequence providing the resultant nucleotide sequence codes for a polypeptide as described above, with the exception of antisense constructs which clearly will not encode a polypeptide.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses for use in the present invention, nucleotide sequences that are capable of hybridising selectively to the H. sapiens Lmo2 sequence, or any variant, fragment or derivative thereof, or to the complement of any of the above. Nucleotide sequences are preferably at least 15 nucleotides in length, more preferably at least 20, 30, 40 or 50 nucleotides in length.

Polynucleotides for use in the invention capable of selectively hybridising to the Lmo2 nucleotide sequences presented in Royer-Pokora et al., 1991, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred polynucleotides for use in the invention will comprise regions homologous to nucleotides 86 to 248 and/or nucleotides 277 to 448 preferably at least 80 or 90% and more preferably at least 95% homologous to nucleotides 86 to 242 and/or nucleotides 283 to 448.

The term “selectively hybridisable” means that the polynucleotide used as a probe is used under conditions where a target polynucleotide of the invention is found to hybridise to the probe at a level significantly above background. The background hybridisation may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical polynucleotide sequences while an intermediate (or low) stringency hybridisation can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0).

Where the polynucleotide of the invention is double-stranded, both strands of the duplex, either individually or in combination, are encompassed for use in the present invention. Where the polynucleotide is single-stranded, it is to be understood that the complementary sequence of that polynucleotide is also included within the scope of the present invention.

Polynucleotides which are not 100% homologous to the sequences used in the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the H. sapiens Lmo2 sequence under conditions of medium to high stringency. Similar considerations apply to obtaining species homologues and allelic variants of human Lmo2.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Binding Partners

Lmo2 forms multiprotein complexes with and interacts with other cellular proteins. Such proteins are termed herein “functionally related proteins”. Clearly, targeting of these Lmo2 binding partners and/or an interaction between Lmo2 and a binding partner thereof may be used to modulate Lmo2-mediated angiogenesis.

Several binding partners have already been identified:

TAL1 is a basic helix-loop-helix (bHLH) protein that has been shown to form complexes with Lmo2 in erythroid cells (Valge-Archer et al., 1994; Wadman et al., 1994). TAL2 and LYL1 are also bHLH proteins that have been shown to form complexes with Lmo2 in vivo (Wadman et al., 1994). Lmo2 binds to TAL1, TAL2 and LYL1 but not to other bHLH proteins such as E47, Id1, MyoD, MYC and MAX. However, Lmo2 can bind to ready assembled heterodimers between TAL1 and other bHLH proteins such as E47, such that they are involved in forming a single multiprotein complex. They are thus functionally related. The bHLH domains of TAL1, TAL2 and LYL1 are highly related to one another but much less so to other bHLH proteins.

Other proteins identified as binding partners of Lmo2 include LDB1 (Valge-Archer et al., 1998). Further binding partners may be identified using screening methods well known in the art such as two hybrid screens (see below) and immunoprecipitation studies.

Reference to binding partners includes homologues, derivatives and fragments thereof as described above for Lmo2. Preferred fragments typically comprise the bHLH domains of TAL1, TAL2 or LYL1.

B. Assays for Substances Capable of Affecting Lmo2/Binding Partner Expression or Function

Our results demonstrate a role for Lmo2 in angiogenesis. As discussed above, angiogenesis plays an important role in disease processes. Consequently, one possible means of treating or preventing a disease associated with angiogenesis, such as solid tumours, would be to administer to a patient in need of such treatment an effective amount of a substance that downregulates Lmo2 expression or affects Lmo2 function in a relevant tissue. An example of such a substance would be an antisense Lmo2 construct or an Lmo2-derived peptide.

The present invention provides assay that are suitable for identifying substances that bind to Lmo2 polypeptides or binding partners thereof (reference to which includes homologues, variants, derivatives and fragments as described above). In addition, assays are provided that are suitable for identifying substances that interfere with Lmo2 binding to other components of the cell, such as other polypeptides. Such assays are typically in vitro. Assays are also provided that test the effects of candidate substances identified in preliminary in vitro assays on intact cells in whole cell assays and/or in vivo.

A substance that inhibits Lmo2-mediated angiogenesis as a result of an interaction with Lmo2 polypeptides may do so in several ways. It may directly disrupt the binding of Lmo2 to a component of a multiprotein complex by, for example, binding to Lmo2 and masking or altering the site of interaction with the other component. Candidate substances of this type may conveniently be preliminarily screened by in vitro binding assays as, for example, described below and then tested, for example in a whole cell assay as described below. Examples of candidate substances include antibodies which recognise Lmo2.

A substance which can bind directly to Lmo2 may also inhibit its function in angiogenesis by altering its subcellular localisation thus preventing Lmo2 and its binding partners from coming into contact within the cell. This can be tested using, for example the whole cells assays described below. Non-functional homologues of Lmo2 may also be tested for inhibition of angiogenesis since they may compete with Lmo2 for binding to other cellular components whilst being incapable of the normal functions of Lmo2 or block the function of Lmo2 bound its cellular target. Such non-functional homologues may include naturally occurring Lmo2 mutants and modified Lmo2 sequences or fragments thereof. In particular, fragments of Lmo2 which comprise either or both LIM domain but lack other functional domains may be used to compete with full length Lmo2 for binding to components of multiprotein complexes.

Alternatively, instead of preventing the association of the components directly, the substance may suppress the biologically available amount of Lmo2. This may be by inhibiting expression of the component, for example at the level of transcription, transcript stability, translation or post-translational stability. An example of such a substance would be antisense RNA or double-stranded interfering RNA sequences which suppresses the amount of Lmo2 mRNA biosynthesis.

Other suitable substances may be identified by screening methods of the invention. For example, a candidate substance whose activity it is desired to test may be administered to a cell which expresses Lmo2 in the absence of the candidate substance. Such screening methods may also be performed in vivo on intact multicellular animals, such as mammals, for example mice, rats or hamsters or by using cell lines.

The present invention also provides assays that are suitable for identifying substances that bind to Lmo2 polypeptides. Such assays are typically in vitro. Assays are also provided that test the effects of candidate substances identified in preliminary in vitro assays on intact cells in whole cell assays and/or in intact multicellular animals.

Candidate Substances

Suitable candidate substances include peptides, especially from about 5 to 30 or 10 to 25 amino acids in size, based on the sequence of the various domains of Lmo2 or binding partners thereof, or variants of such peptides in which one or more residues have been substituted. Peptides from panels of peptides comprising random sequences or sequences which have been varied consistently to provide a maximally diverse panel of peptides may be used. Other candidate substances include antibodies. In particular, intracellularly expressed antibodies such as scFvs that bind to Lmo2 or a binding partner thereof may be identified using an antibody-antigen two hybrid screen where, for example, a library of scFvs is expressed in cells that comprise (i) a reporter construct (ii) a construct that expresses Lmo2 fused to a DNA binding domain and (iii) a construct that expresses TAL1 fused to a transcriptional activation domain. Antibodies that inhibit Lmo2/TAL1 binding will reduce the signal produced from the reporter construct (such as green fluorescent protein, beta-galactosidase, luciferase or CAT). Two hybrid screen technology is known in the art.

Combinatorial libraries, peptide and peptide mimetics, defined chemical entities, oligonucleotides, and natural product libraries may be screened for activity as modulators of Lmo2 expression and/or function. The candidate substances may be used in an initial screen in batches of, for example 10 substances per reaction, and the substances of those batches which show inhibition tested individually. Candidate substances which show activity in in vitro screens such as those described below can then be tested in whole cell systems, such as those described below which will be exposed to the inhibitor and tested for modulation of

Lmo2 Function and/or Expression, in Particular Via Effects on Angiogenesis.

Lmo2/Binding Partners Binding Assays

One type of assay for identifying substances that bind to Lmo2 or binding partners thereof, possibly interfering with its function, synthesis, trafficking and/or degradation in vivo, involves contacting a Lmo2 polypeptide or binding partner thereof, which is immobilised on a solid support, with a non-immobilised candidate substance determining whether and/or to what extent the Lmo2 polypeptide or binding partner thereof and candidate substance bind to each other. Alternatively, the candidate substance may be immobilised and the Lmo2 polypeptide or binding partner thereof non-immobilised.

In a preferred assay method, the Lmo2 polypeptide or binding partner thereof is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads. As a control, binding of the candidate substance, which is not a GST-fusion protein, to glutathione-agarose beads (and/or a GST only control) is determined in the absence of the Lmo2 polypeptide or binding partner thereof. The binding of the candidate substance to the immobilised Lmo2 polypeptide or binding partner thereof is then determined. This type of assay is known in the art as a GST pulldown assay. Again, the candidate substance may be immobilised and the Lmo2 polypeptide or binding partner thereof non-immobilised.

It is also possible to perform this type of assay using different affinity purification systems for immobilising one of the components, for example Ni-NTA agarose and histidine-tagged components.

Binding of the Lmo2 polypeptide or binding partner thereof to the candidate substance may be determined by a variety of methods well-known in the art. For example, the non-immobilised component may be labelled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml.

Polypeptides that interact with Lmo2, including components of multimeric protein complexes involving Lmo2, may also be identified by, for example, a two-hybrid screen. The two-hybrid system was developed in yeast (Chien et al., 1991, Proc. Natl. Acad Sci USA 88, 9578-9582) and is based on functional in viva reconstitution of a transcription factor which activates a reporter gene. Specifically, a polynucleotide encoding a protein that interacts with Lmo2 is isolated by: transforming or transfecting appropriate host cells with a DNA construct comprising a reporter gene under the control of a promoter regulated by a transcription factor having DNA a binding domain and an activating domain; expressing in the host cells a first hybrid DNA sequence encoding a first fusion of part or all of Lmo2 and either the DNA binding domain or the activating domain of the transcription factor; expressing in the host cell a library of second hybrid DNA sequences encoding second fusion of part or all putative Lmo2 binding proteins and the DNA binding domain or activating domain of the transcription factor which is not incorporated in the first fusion; detecting binding of an Lmo2 interacting protein to Lmo2 in a particular host cell by detecting the production of reporter gene product in the host cell; and isolating second hybrid DNA sequences encoding the interacting protein from the particular host cell. Presently preferred for use in the assay are a lexA promoter to drive expression of the reporter gene, the lacZ reporter gene, a transcription factor comprising the lexA DNA binding domain and the GAL4 transactivation domain, and yeast host cells.

Several binding partners of Lmo2 have already been identified as described in section A (i.e. TAL1, TAL2, LYL1 and LDB1). These known binding partners and/or subsequently identified binding partners may be used to identify substances that affect an interaction between Lmo2 and a binding partner thereof. This may have particular physiological significance since Lmo2 is thought to be a bridging molecule in the assembly of DNA binding complexes such as a complex comprising TAL1, E47, GATA-1 and Ldb1/NL1 (Wadman et al., 1997).

Assays for identifying compounds that modulate interaction of Lmo2 with other proteins may involve transforming or transfecting appropriate host cells with a DNA construct comprising a reporter gene under the control of a promoter regulated by a transcription factor having a DNA-binding domain and an activating domain; expressing in the host cells a first hybrid DNA sequence encoding a first fusion of part or all of Lmo2 and the DNA binding domain or the activating domain of the transcription factor; expressing in the host cells a second hybrid DNA sequence encoding part or all of a protein that interacts with Lmo2 and the DNA binding domain or activating domain of the transcription factor which is not incorporated in the first fusion; evaluating the effect of a test compound on the interaction between Lmo2 and the interacting protein by detecting binding of the interacting protein to Lmo2 in a particular host cell by measuring the production of reporter gene product in the host cell in the presence or absence of the test compound; and identifying modulating compounds as those test compounds altering production of the reported gene product in comparison to production of the reporter gene product in the absence of the modulating compound. An example of a suitable configuration for use in the assay is a lexA promoter to drive expression of the reporter gene, a lacZ reporter gene, the lexA DNA binding domain (or the GAL4 DNA binding domain) fused to Lmo2, the GAL4 transactivation domain fused to the interacting protein, and yeast host cells.

Another type of assay for identifying compounds that modulate the interaction between Lmo2 and an interacting protein involves immobilising Lmo2 or a natural Lmo2 interacting protein, detectably labelling the nonimmobilised binding partner, incubating the binding partners together and determining the effect of a test compound on the amount of label bound wherein a reduction in the label bound in the present of the test compound compared to the amount of label bound in the absence of the test compound indicates that the test agent is an inhibitor of Lmo2 interaction with the protein. Conversely, an increase in the bound in the presence of the test compared to the amount label bound in the absence of the compared indicates that the putative modulator is an activator of Lmo2 interaction with the protein.

Further assays for Lmo2 function include DNA binding assays such as electrophoretic mobility shift assays (EMSAs). Wadman et al. (1997) have shown that a complex comprising Lmo2 binds to a unique bipartite DNA motif comprising an E-box, CAGGTG, followed approximately 9 basepairs downstream by a GATA site. Suitable methodology is described in Wadman et al. (1997). Thus the present invention provides a method for determining whether a candidate substance modulates Lmo2 mediated angiogenesis by determining whether the candidate substance modulates binding of a multiprotein complex comprising Lmo2 to a double stranded DNA molecule comprising a cognate recognition site.

Whole Cell Assays

Candidate substances may also be tested on whole cells for their effect on the expression and/or function of Lmo2 or a binding partner thereof. Preferably the candidate substances have been identified by the above-described in vitro methods. Alternatively, rapid throughput screens for substances capable of modulating Lmo2/binding partner function may be used as a preliminary screen and then used in the in vitro binding assay described above to confirm that the affect is on Lmo2 or the binding partner.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Suitable whole cells for testing inhibition, are those which express Lmo2 such as vascular endothelial cells, MEL (erythroid), 2114 (mouse T cell thymoma) or KOPT1 (human T-ALL cell line with t(11; 14)(p13;q11) activating Lmo2).

Typically, an assay to determine the effect of a candidate substance, which may have been identified by an in vitro method of the invention, on the expression and/or function of Lmo2 or a binding partner thereof comprises administering the candidate substance to a cell and determining whether the substance inhibits or reduces, or enhances the expression and/or function of Lmo2 or a binding partner thereof.

Lmo2/binding partner expression may be determined by measuring mRNA and/or protein levels as using techniques such as quantitative RT-PCR and western blotting. A candidate substance is typically considered to be an inhibitor of Lmo2 expression if Lmo2 expression is reduced to below 50%, preferably below 40, 30, 20 or 10% of that observed in untreated control cells. A candidate substance is typically considered to enhance Lmo2 expression if Lmo2 expression is reduced to below 50%, preferably below 40, 30, 20 or 10% of that observed in untreated control cells.

Lmo2/binding partner function may be measured by, for example, an in vivo assay for angiogenesis as described below.

The concentration of candidate substances used will typically be such that the final concentration in the cells is similar to that described above for the in vitro assays.

The types of assays performed on whole cells described above may also be performed on intact multicellular animals, typically mammals such as rodents, pigs or non-human primates. However generally, assays performed on intact multicellular animals will be of the types described below in section C.

C. In Vivo Angiogenesis Assays

A particularly preferred in vivo test involves determining the effect of a candidate substance on tumour angiogenesis in a mammal, such as mice, that expresses an Lmo2 polypeptide or binding partner thereof fused to a detectable marker such as a histologically detectable marker (for example lacZ). The mice are typically transgenic for the Lmo2-marker fusion.

Tumours may be induced in the animals in several ways. For example, a carcinogenic substance may be administered to the animal to induce tumour formation. Alternatively, an oncogenic virus, such as murine leukaemia virus, may be administered to the mouse. MLV induces thymomas in mice. Thymomas are particularly suitable in the context of the present invention for measuring tumour angiogenesis.

Another technique for inducing tumours is to use animals that have a genetic predisposition to tumours. Examples include p53-null mice and p53 gain of function mice that typically develop thymomas and/or epithelial tumours such as lung or kidney tumours that are especially likely to metastasise. Other examples include animals with a T-cell oncogene such as cd2-lmo2 gain of function which leads to thymoma development. These genetic methods may be used in conjunction with carcinogenic substances.

In a preferred embodiment, tumour tissue from an animal as described above that has developed a suitable tumour growth may be implanted into one or more animals that lack the tumour, in particular nude or SCID mice.

In an in vivo assay of the invention, a candidate substance is administered to an animal and the effect on angiogenesis, such as tumour angiogenesis, determined. This may typically involve determining the amount of vascularisation over a period of time, for example by measuring the amount of tissue expressing an Lmo2-reporter fusion polypeptide. Also, an effect on tumour growth in treated animals versus untreated animals may be measured by, for example measuring the change in tumour size over time.

Several other assay models for inducing angiogenesis in vivo have been developed.

(i) The corneal pocket assay involves the surgical implantation of polymer pellets containing angiogenic factors in the cornea of larger animals such as rabbits.

(ii) The chick chorioallantoic membrane (CAM) assay involves the removal and transfer of a chick embryo from the shell to a cup. The angiogenic material is dried on a glass cover slip and placed on the chorioallantoic membrane and the appearance of new vessels is observed.

(iii) The rabbit ear chamber assay requires the surgical insertion of a glass or plastic viewing device and measurement of capillary migration by microscopy.

(iv) The rat dorsal air sac assay involves implants of stainless steel chambers containing angiogenic factors.

(v) An alginate assay which generates an angiogenic response has been described which involves the injection of tumour cells encased in alginate subcutaneously into mice. The accumulation of haemoglobin in the injected gel is used to quantitate the angiogenic response.

The CAM assay (Ausprunk et al., 1975, Am. J. Pathol., 79:597-618 and Ossonski et al., 1980, Cancer Res., 40:2300-2309) measures angiogenesis in the chick chorioallantoic membrane (CAM). The CAM assay has been used to measure both angiogenesis and neovascularisation of tumour tissues.

The CAM assay is a well recognised assay model for in vivo angiogenesis because neovascularisation of whole tissue is occurring, and actual chick embryo blood vessels are growing into the CAM or into the tissue grown on the CAM.

The CAM assay illustrates inhibition of neovascularisation based on both the amount and extent of new vessel growth. Furthermore, it is easy to monitor the growth of any tissue transplanted upon the CAM, such as a tumour tissue. Finally, the assay is particularly useful because there is an internal control for toxicity in the assay system. The chick embryo is exposed to any test reagent, and therefore the health of the embryo is an indication of toxicity.

The corneal pocket assay (D'Amato, et al., 1994. Proc. Natl. Acad. Sci., 91:4082-4085) performed on rabbit eyes has been used to measure both angiogenesis and neovascularisation in the presence of angiogenic inhibitors such as thalidomide.

The rabbit eye assay is a well recognised assay model for in vivo angiogenesis because the neovascularisation process, exemplified by rabbit blood vessels growing from the rim of the cornea into the cornea, is easily visualised through the naturally transparent cornea of the eye. Additionally, both the extent and the amount of stimulation or inhibition of neovascularisation or regression of neovascularisation can easily be monitored over time.

The chimeric mouse assay (Yan, et al., 1993, J. Clin. Invest. 91:986-996) measures angiogenesis in a chimeric mouse:human mouse model.

The chimeric mouse assay is a useful assay model for in vivo angiogenesis because the transplanted skin grafts closely resemble normal human skin histologically and neovascularisation of whole tissue is occurring wherein actual human blood vessels are growing from the grafted human skin into the human tumour tissue on the surface of the grafted human skin. The origin of the neovascularsation into the human graft can be demonstrated by immunohistochemical staining of the neovasculature with human-specific endothelial cell markers.

The chimeric mouse assay may be used to assess regression of neovascularisation based on both the amount and extent of regression of new vessel growth. Furthermore, it is easy to monitor effects on the growth of any tissue transplanted upon the grafted skin, such as a tumour tissue.

A further in vivo test described in U.S. Pat. No. 5,382,514 comprises providing a liquid matrix material which forms a matrix gel when injected into a host; adding an angiogenic agent to the liquid matrix material; injecting the liquid matrix material containing the angiogenic agent into a host to form a matrix gel; recovering the matrix gel from the host; and quantitating angiogenesis of the recovered matrix gel.

In the assay method of the invention, the in vivo test described in U.S. Pat. No. 5,382,514 would typically be performed in the presence and absence of a candidate substance and the effect of the candidate compound on the modulation of angiogenesis determined, such as the degree of inhibition or stimulation of angiogenesis.

D. Uses of Modulators of Angiogenesis

Where the growth of new blood vessels is the cause of, or contributes to, the pathology associated with a disease, inhibition of angiogenesis will reduce the deleterious effects of the disease. Examples include rheumatoid arthritis, diabetic retinopathy, inflammatory diseases, restenosis, and the like. Where the growth of new blood vessels is required to support growth of a deleterious tissue, inhibition of angiogenesis will reduce the blood supply to the tissue and thereby contribute to reduction in tissue mass based on blood supply requirements.

Examples include growth of tumours where neovascularisation is a continual requirement in order that the tumour grow beyond a few millimetres in thickness, and for the establishment of solid tumour metastases.

As described earlier, angiogenesis includes a variety of processes involving neovascularisation of a tissue including “sprouting”, vasculogenesis, or vessel enlargement, all of which angiogenesis processes are mediated by and dependent upon the expression of Lmo2. With the exception of traumatic wound healing, corpus leuteum formation, the menstrual cycle and embryogenesis, it is believed that the majority of angiogenesis processes are associated with disease processes.

There are a variety of diseases in which angiogenesis is believed to be important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as pre-neoplastic lesions (e.g. in the head, lung or neck), solid tumours, solid tumour metastases, angiofibromas, retrolental fibroplasia, hemangiomas, Kaposi sarcoma and other cancers which require neovascularisation to support tumour growth.

Thus, methods which inhibit angiogenesis in a diseased tissue ameliorate symptoms of the disease and, depending upon the disease, can contribute to treating the disease. In one embodiment, the invention contemplates inhibition of angiogenesis, per se, in a tissue. The extent of angiogenesis in a tissue, and therefore the extent of inhibition achieved by the present methods, can be evaluated by a variety of methods.

As described herein, any of a variety of tissues, or organs comprised of organised tissues, can support angiogenesis in disease conditions including skin, muscle, gut, connective tissue, joints, bones and other tissues in which blood vessels can invade upon angiogenic stimuli.

Thus, in one related embodiment, the tissue is an inflamed tissue and the angiogenesis to be inhibited is inflamed tissue angiogenesis where there is neovascularisation of inflamed tissue.

In this class the method contemplates inhibition of angiogenesis in arthritic tissues, such as in a patient with chronic articular rheumatism, in immune or non-immune inflamed tissues and in psoriatic tissue.

In another related embodiment, the tissue is a retinal tissue of a patient with diabetic retinopathy, macular degeneration or neovascular glaucoma and the angiogenesis to be inhibited is retinal tissue angiogenesis where there is neovascularisation of retinal tissue.

In an additional related embodiment, the tissue in which angiogenesis is to be inhibited is a tumour tissue of a patient with a solid tumour, metastases, a skin cancer, a breast cancer, a hemangioma or angiofibroma, and the angiogenesis to be inhibited is tumour tissue angiogenesis where there is neovascularisation of a tumour tissue. Typical solid tumour tissues treatable by the present methods include lung, pancreas, breast, colon, laryngeal, ovarian, and the like tissues.

Inhibition of tumour tissue angiogenesis is a particularly preferred embodiment because of the important role neovascularisation plays in tumour growth. In the absence of neovascularisation of tumour tissue, the tumour tissue does not obtain the required nutrients, slows in growth, ceases additional growth, regresses and ultimately becomes necrotic resulting in killing of the tumour.

The present method for inhibiting angiogenesis in a tissue comprises contacting a tissue in which angiogenesis is occurring, or is at risk for occurring, with an inhibitor of the invention, such as an antisense Lmo2 construct or an inhibitor identified by the assay methods of the invention

In a further embodiment, angiogenesis may be stimulated by an Lmo2-mediated mechanism such as to allow for inducement of an additional vascular supply, for example, in wounded or ischemic tissue where angiogenesis is needed to restore normal healing and regeneration.

The present method for stimulating angiogenesis in a tissue comprises contacting a tissue in which it is desired to stimulate angiogenesis with a stimulant of the invention, such as a stimulant identified by the assay methods of the invention. It may also be possible to stimulate angiogenesis by administering a composition comprising Lmo2 protein or a nucleic acid construct that directs expression of Lmo2 in the tissue.

E. Administration

Inhibitors of Lmo2/binding partner expression and/or function, such as substances identified or identifiable by the assay methods of the invention, may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular, oral or transdermal administration.

Typically, each substance may be administered at a dose of from 0.01 to 30 mg/kg body weight, preferably from 0.1 to 10 mg/kg, more preferably from 0.1 to 1 mg/kg body weight.

Polypeptides, including antibodies may also be administered using protein transduction systems such as protein transduction systems based on the 3^{rd} helix of the Antennapedia protein.

Polynucleotides/vectors encoding polypeptide components (or antisense constructs) for use in modulating Lmo2 expression and/or function may be administered directly as a naked nucleic acid construct. The polynucleotides/vectors may further comprise flanking sequences homologous to the host cell genome.

When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector according to the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, oral or transdermal administration.

The particular dosage ranges for the administration of the modulator of the invention depend upon the form of the modulator, and its potency, and are amounts large enough to produce the desired effect in which angiogenesis is inhibited or stimulated. The dosage should not be so large as to cause adverse side effects, such as hyperviscosity syndromes, pulmonary oedema and congestive heart failure. Generally, the dosage will vary with the age, condition, sex and extent of the disease in the patient and can be determined by one of skill in the art. The dosage can also be adjusted by the individual physician in the event of any complication.

An effective amount is an amount of an inhibitor of the invention sufficient to produce a measurable modulation of angiogenesis in the tissue to which it is administered, i.e., an angiogenesis-modulating amount. Modulation, such as inhibition or stimulation, of angiogenesis can be measured in situ by immunohistochemistry or by other methods known in the art.

The invention will now be further described by way of Examples, which are meant to serve to assist one of ordinary skill in the art in carrying out the invention and are not intended in any way to limit the scope of the invention.

### EXAMPLES

Methods

Homologous Recombination

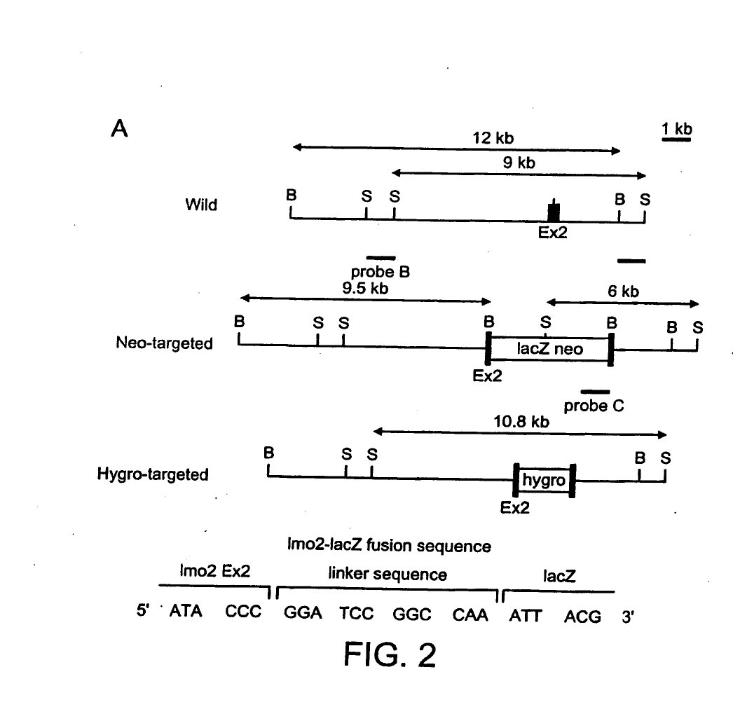

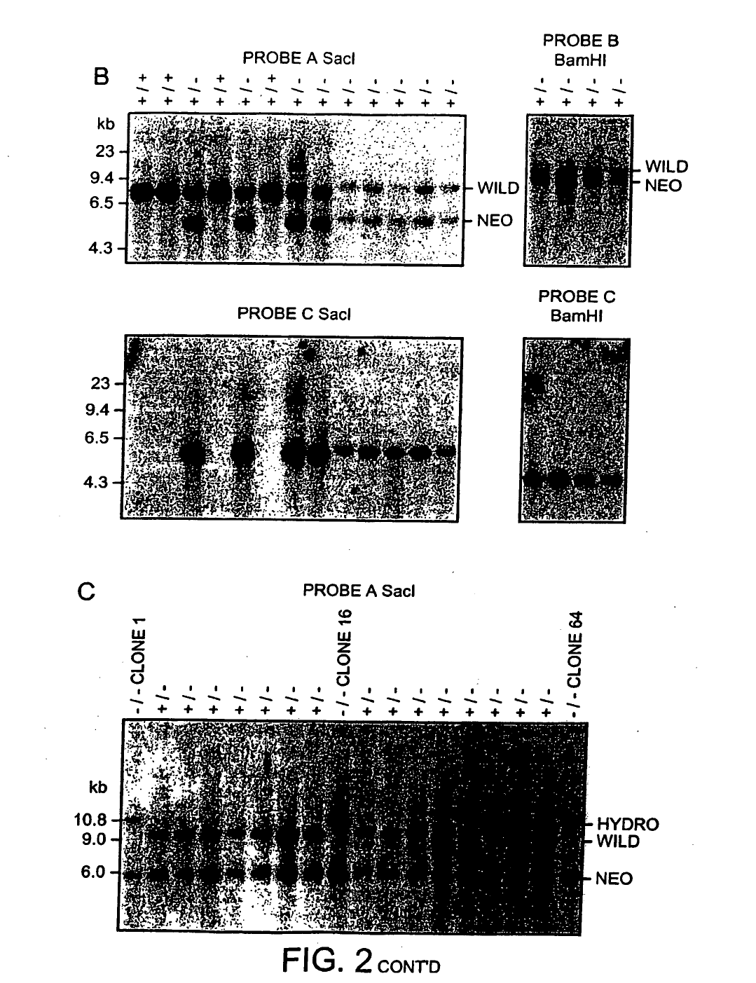

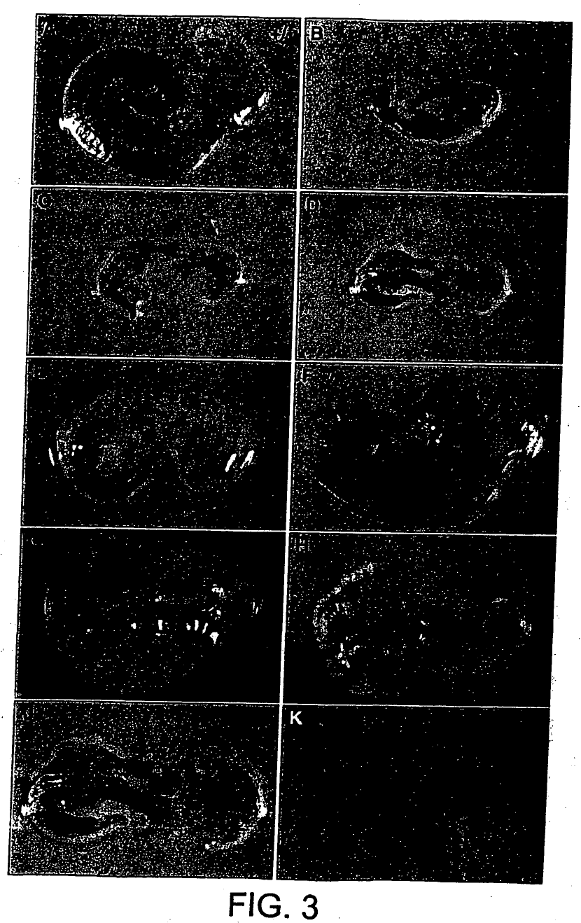

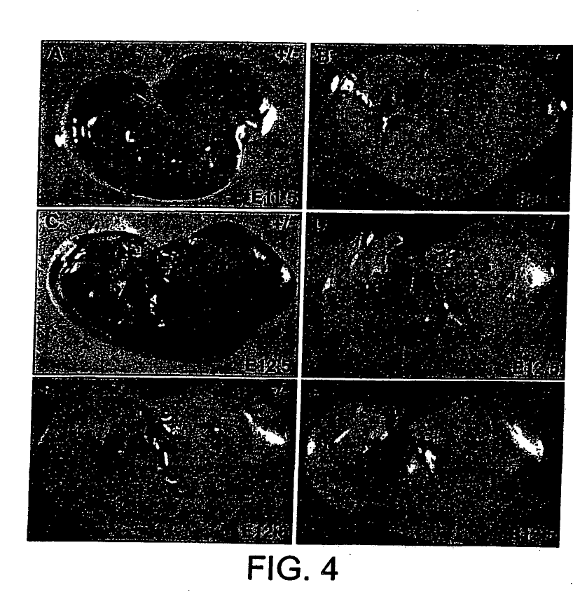

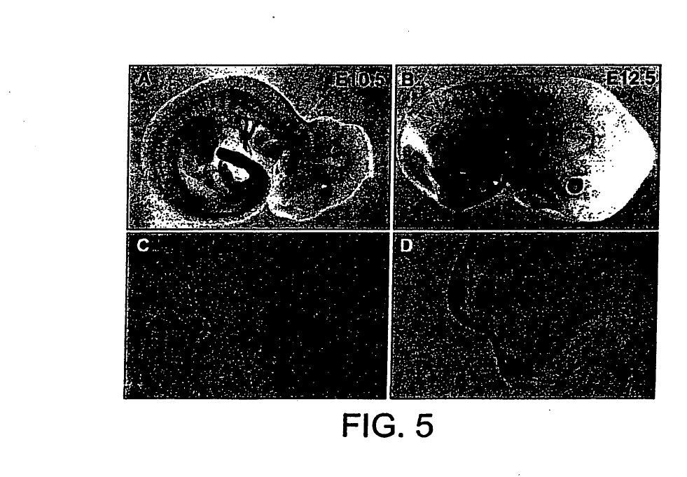

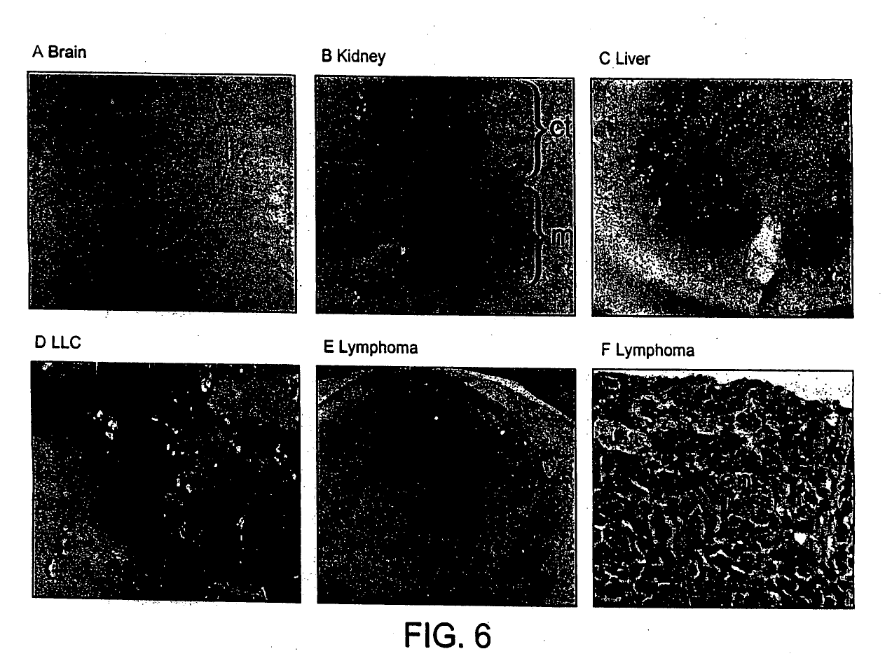

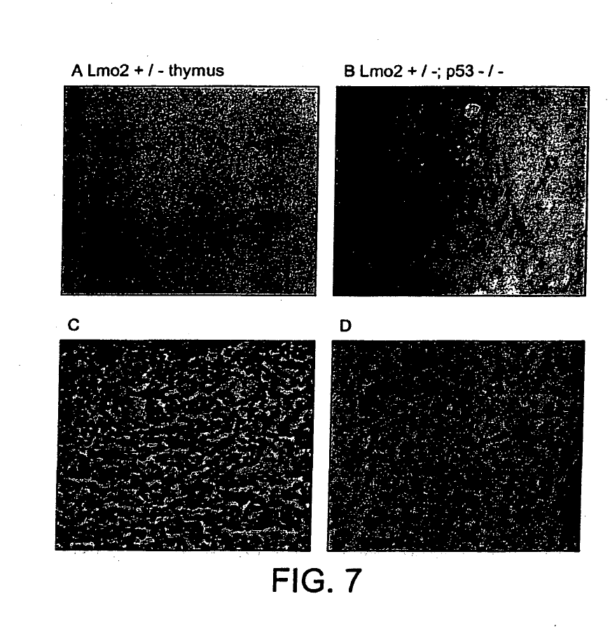

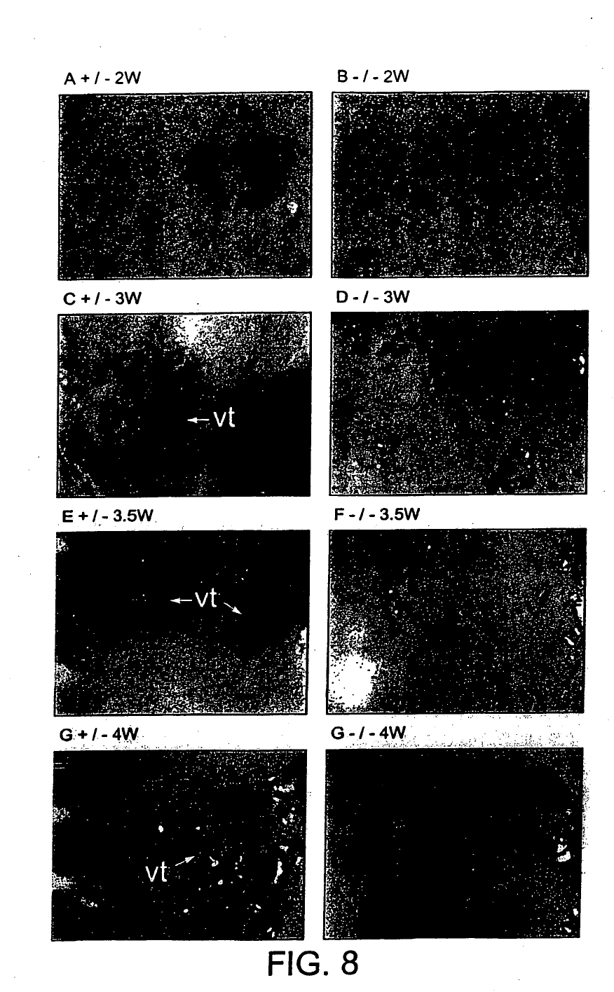

Construction of the Lmo2-lacZ targeting vector is described in FIG. 2. 25 μg of linearised vector DNA was used in the transfection to CCB ES cells by electroporation. Selection of resistant was done either by cell growth in medium containing 400 μg/ml G418 (Gibco) or 300 μg/ml hygromycin B (Calbiochem). Targeted clones were found by sequentially hybridising of ES cell DNA (by standard filter hybridisation (LeFranc et al., 1986) with flanking probes from both sides of the targeting region (see FIG. 2 legend) and confirming the presence of a single insertion of the targeting fragment with an internal probe.

Production of Chimaeric Mice and Germ Line Transmission

ES cells were micro-injected into C57/BL6 blastocysts and transferred to CBA/C57/BL6 recipients. For the embryonic studies, the day of injection was designated as E2.5. Germ-line transmission of the targeted allele was confirmed by Southern filter hybridisation using tail DNA. The embryonic lethal phenotype of homozygous Lmo2 null −/− embryos was confirmed for the KZ26 germ-line transmitted allele by inter-breeding heterozygous +/− carrier mice and examining the resulting embryos. This showed that the KZ26 Lmo2-lacZ null mutation behaves the same as the previously described Lmo2 mutation (Warren et al., 1994).

Whole Mount X-gal Staining

Embryos at each stage were dissected from the uterus and any maternal decidual tissue was removed. Whole embryos were examined for X-gal staining of beta-galactosidase activity (resulting from Lmo2-lacZ gene expression) according to the procedure described in Allen et al., 1988. For histological studies, embryos were fixed in 10% buffered formalin after X-gal staining and embedded in paraffin. Sections were mounted on slides and counter-stained with eosin.

Tumour Growth

In Lmo2 +/−, p53 −/− mice were crossed and subsequently with each other to obtain an experimental group mice with Lmo2 +/−, p53 −/− genotype.

Lewis lung carcinoma (LLC) cells were cultured in DMEM supplemented with 10% foetal calf serum, trypsinised and resuspended in PBS at a concentration of 2.5×10⁵ cells/100 μl. 100 μl of cell suspension was injected into subcutaneous dorsa in the proximal midline of C57B16 Lmo2+/−heterozygous mice. Tumour development was generally first observed at 3 days and the maximum tumour size of 1.7 cm reached at 14 days.

Teratocarcinomas were produced from ES cells implanted in nude mice. ES cells were grown on neomycin-resistant feeders in the presence of LIF prior to trypsinisation. The washed cells were resuspended in PBS at a concentration of 2×10⁶/100 μl. 100 μl of cell suspension was injected subcutaneously into both flanks of adult MF1 female nude mice. A cohort of 50 mice was used for each of Lmo2+/−and Lmo2−/− ES cells. Tumours were measured every 4 days with a dial-caliper, volumes were determined using the formula, width²×length×0.52.

### Example 1

The null mutation of the Lmo2 gene in mouse results in an embryonic lethal phenotype at around embryonic stage E9-10 due to lack of yolk sac erythropoiesis. However, in addition to the expression in developing erythrocytes, Lmo2 is expressed in various other locations and is also important in the development of adult haematopoietic system. The importance of Lmo2 function at these sites of expression cannot be assessed in homozygous Lmo2 null mice due to the lethality of this mutation. As an aid to assess potential roles for Lmo2 after E10, we have introduced the lacZ gene, by homologous recombination, into the Lmo2 gene in ES cells. FIG. 2A shows the structure of the Lmo2-lacZ fusion gene with lacZ incorporated into exon2 of Lmo2. An ES clone was selected with the lacZ gene knock-in of one Lmo2 allele (FIG. 2A, KZ26 +/− ES cells) and this clone was re-transfected with a Lmo2-hygromycin vector to create ES cells with a second targeted Lmo2 allele (KZ26 −/−; three independent clones were studied, clones 1, 16 and 64). Expression of beta-galactosidase is controlled by the Lmo2 gene in these ES cells or their derivatives in vivo after injection of the ES cells into blastocysts and generation of embryos. In addition, the Lmo2 +/− ES cell (KZ26) was used to obtain germ-line transmission of the targeted allele to give KZ26 heterozygous mice.

We have used beta-galactosidase to follow the fate of Lmo2-expressing ES cells in developing embryos. Initially we used the KZ26 heterozygous mice to study the pattern of beta-galactosidase expression from E 8.5 to E 14.5. During these embryonic stages, we found X-gal staining chiefly in endothelial cells of the whole body vascular system. From E 11.5 it was also found just beneath the apical ectodermal ridge of limb buds, which is mesenchymal tissue called the progress zone. This is an a vascular area and the field of sprouting angiogenesis. Additional sites were the tail bud and hippocampus (FIGS. 3A and 3B show E10.5 and E12.5 Lmo2+/−embryos respectively; expression of beta-galactosidase in the limb buds can be seen in histological sections, FIG. 3D; the X-gal staining of vessel endothelium is shown in a tissue section (FIG. 3C)). These beta-galactosidase expression data are compatible with Lmo2 expression patterns observed with RNA in situ hybridisation indicating that the expression of beta-galactosidase in endothelial cells is also a true reflection of Lmo2 promoter activity in embryogenesis.

As a method of following the fate of ES cells with the Lmo2-lacZ fusion gene and studying the consequence of the homozygous null mutation, we have compared the beta-galactosidase staining patterns of chimaeric embryos derived from injection of KZ26 +/− and −/− ES cells into blastocysts (FIG. 4). A number of findings emerged from this study. While at E9.5, there was no obvious difference between +/− and −/− chimaeric mice (data not shown), when E11.5 embryos of comparable size were stained with X-gal from either KZ26 +/− or −/− injections (FIGS. 4 A and B respectively), the beta-galactosidase patterns were markedly different with respect of endothelial cell staining. KZ26 +/− E11.5 chimaeric embryos have a staining pattern very like that of heterozygous embryos (FIG. 3). On the other hand, the E11.5 −/− chimaeric embryos showed almost no contribution of ES-derived cells to vascular system (FIG. 3B), although ES cell contribution to limb buds and hippocampus was retained. Peripheral scattered endothelial cells were observed by X-gal staining but there was no contribution of Lmo2 −/− ES cells to major vessel endothelial cells. The consistency of this observation was shown by examination of KZ26 +/− and −/− embryos at E12.5. FIG. 4C shows a KZ26+/−chimaeric embryo with an extensive staining pattern of endothelial cells and in addition, limb bud and hippocampal staining.

By contrast, E12.5 KZ26 −/− embryos from clone 1 (3D), clone 16 (3E) or clone 64 (3F) did not have endothelial cell staining but each had comparable limb bud, tail and hippocampal staining. This lack of E12.5 endothelial cell staining parallels that of the E11.5 −/− chimaeric embryos (FIG. 4B).

Vascular endothelial remodelling begins at around E10.5 in mice. The effect of null Lmo2 mutation was studied at this crucial stage. Embryos were obtained at E10.5 from a litter resulting from injection of KZ26 −/− clone 1 into blastocysts and whole mount stained with X-gal. Seven embryos were compared in their staining pattern (FIG. 5, panels B-H) with an E10.5 KZ26 +/− chimaera (FIG. 5, panel A). In the −/− chimaeras, there was remarkable size variation which was inversely proportional with the degree of ES cell contribution to endothelial cells (as judged by X-gal staining). Mice with high X-gal staining (i.e. high contribution of Lmo2 −/− ES-derived cells) were smaller than those with low contribution in the same litters (FIG. 5). Normal size Lmo2 −/− embryos had essentially no endothelial cells stained, while retaining limb bud and tail staining. In those small mice with high X-gal staining, there was no well organised vascular system (FIG. 5, panel J, K) suggesting that when the chimaerism of −/− ES cells was high, the endothelial remodelling fails due to lack of Lmo2 and these embryos are consequently destined to die (see below, Table 1). If there is relatively low chimaerism in the embryos, they survive presumably because blastocyst-derived cells can replace ES-derived ones in the re-modelling process.

These data indicate a selective inability of Lmo2-null ES cells to contribute to endothelial cells after about E10.5-E11.5. The consistency of these observations have been confirmed by analysis of a large number of embryos from litters made by injecting the KZ26 −/− clones into blastocysts. These data are summarised in Table 1. Whilst the chimaeric embryos made with the heterozygous ES cells KZ26 uniformly survived at all stages through E9.5 to E12.5 (Table 1A), survival of the KZ26 −/− chimaeras was only 100% at E9.5. Thereafter, viability progressively decreased, concomitant with lower proportions of lacZ-positive chimaeras. All together, 188 −/− chimaeric embryos were analysed and a large gap in the survival ratio and X-gal-positive ratio occurs between E10.5 and E11.5. There was no such relationship in +/− chimaeric mice. Endothelial remodelling and survival do not occur if embryos have high proportions of Lmo2 −/− ES-cell derivatives due to the failure to form mature vascular system. It is possible that Lmo2 −/− endothelial cells become selectively apoptotic after E10.5.

Our data show a selective failure of Lmo2-null ES cells to contribute to vascular endothelial cells after about E10.5-E11.5. Thus the initial process of vasculogenesis does not appear to require Lmo2 but Lmo2 is necessary for angiogenesis during mouse development. In previous studies, Lmo2 was shown to be necessary for the initiation of definitive haematopoiesis. These data together suggest that at least two different functions of Lmo2 must now be considered viz. a function in both angiogenesis and haematopoiesis. It is of significant interest that the Tal1/Scl gene has a similar role in yolk sac angiogenesis and in primitive and definitive haematopoiesis. The parallel between these effects of Lmo2 and Tal1 is presumably due to the interaction between these two proteins in which the Tal1, in conjunction with E47, forms a DNA-binding element and Lmo2 acts as a protein-protein bridging molecule, through its LIM domains. A plausible explanation for the Lmo2-Tal1 concordance in distinct embryological functions is that the Lmo2 molecule brings different sets of proteins together with the Tal1-E47 in different developmental cell types. This would make different protein complexes in different sites of function (e.g. in haematopoiesis and angiogenesis) but still have in common the Lmo2 and Tal1 proteins. The variation in composition of these putative complexes is worthy of investigation, as are the target genes (positively or negatively regulated) for these different complexes. Moreover, several tyrosine kinase-type cell surface receptors are specifically expressed on endothelial cells and they have important roles in either vasculogenesis or angiogenesis.

### Example 2

The results in Example 1 demonstrate that that Lmo2 is required for re-modelling of the vascular endothelium in mouse development. A corollary of this is that adult angiogenesis is also dependent on Lmo2. Since tumour growth is dependent on oxygenation through the development of a blood supply (Hanahan et al., (1996) Cell 86:353), this requirement for Lmo2 in angiogenesis implies that the protein would be a vital factor in tumour neo-vascular formation. This possibility has been examined using Lmo2 null ES cells and using Lmo2 +/− mice.

We have studied Lmo2 expression via β-galactosidase in blood vessel endothelium on heterozygous Lmo2 mice (Lmo2 +/−). Excluding tissues with high endogenous β-galactosidase activity such as salivary gland, Lmo2 expression was traced in situ by X-gal staining. Thymus, brain, liver and kidney were dissected from 12 weeks old Lmo2+/−mice and wild-type litter mates, and pieces used for whole mount X-gal staining. Negligible endogenous β-galactosidase activity was observed in the endothelial cells of these organs of wild-type mice and blue staining in Lmo2+/−mice was attributed to Lmo2 gene expression. Low levels of vascular system staining was found in Lmo2+/−mice. FIG. 6 shows expression and localisation of β-galactosidase in brain blood vessels (FIG. 6A), in glomeruli of kidney cortex and vessels of medulla (FIG. 6B) and in the liver vascular system (FIG. 6C).

### Example 3

The comparison of β-galactosidase levels in normal tissue endothelium and tumour endothelium revealed a marked up-regulation of Lmo2 expression in the latter. Lmo2 expression in tumour vessels was studied in solid tumours developed with Lewis lung carcinoma (LLC) cells implanted subcutaneously into 12 weeks old Lmo2 +/− mice or wild-type controls. Tumours were dissected 2 weeks after implantation for whole mount X-gal staining. Strong β-galactosidase activity was seen only in LLC tumour vessels of Lmo2 +/− mice (FIG. 6D; no activity was found in tumours on wild-type mice). Secondly, Lmo2 heterozygous mice were crossed with the tumour-prone p53 knock-out mice (p53 −/−; Rabbitts, T. H., (1998)) to obtain spontaneous tumours (often thymoma) with Lmo2 controlling β-galactosidase expression. This resulted in tumour vasculature with marked elevation of Lmo2 expression. An example (shown in FIGS. 6E and F) is an abdominal T cell lymphoma in an Lmo2 +/−; p53 −/− mouse showing high levels of lacZ activity in tumour vasculature in a haemorrhagic lymphoma whole mount (FIG. 6E) and specific endothelial staining in histological sections (FIG. 6F). Note that the malignant T cells in this lymphomatous mass are not expressing α-galactosidase (FIG. 6F).

The change of Lmo2 expression during thymoma development was studied using two spontaneous thymomagenesis models. Mice were bred to produce the genotypes Lmo2 +/−; p53 −/− as above and a second line of mice was produced with heterozygous Lmo2 combined with an Lmo2 transgene (CD2-rbtn2: Fisch et al., 1992) which specifies Lmo2 expression in thymus causing clonal T cell leukaemia with lymphoma (Larson et al., (1994); Larson et al., (1995); Larson et al., (1996)). Thymomas developed with long latency in both sets of mice (total of 9 cases in Lmo2 +/−; p53 −/− mice and two cases of Lmo2 +/− with the CD2-rbtn2 transgene). When signs of ill-health indicated lymphomatous deposits of sufficient size, the mice were sacrificed and thymomas subjected to whole mount staining. These were compared with normal thymus staining from 12 week old Lmo2 +/−mice. Some staining was observed in blood vessels of Lmo2 +/− thymus when macroscopically studied (FIG. 7A) and histology showed that this weak staining resides in endothelial cells (FIG. 7D). The blood vessel staining in both Lmo2 +/−; p53 −/− and Lmo2 +/−; CD2-rbtn2 thymomas is much more marked at the macroscopic level (FIGS. 7 B and C, respectively) which accompanies an increase of vascular density. The augmented level of Lmo2 expression is confirmed by histological examination of endothelial cell lacZ expression (FIGS. 7 E and F).

These data suggest that Lmo2 transcription is activated during the process of tumour neo-vascularisation, implying a role in the necessary process of angiogenesis in solid tumour growth. The latter function for Lmo2 was further analysed using a model for solid tumour development in which ES cells cause teratocarcinomas after subcutaneous implantation in immunodeficient nude mice. Teratocarcinomas were made by subcutaneous implantation of either Lmo2 +/−or Lmo2 −/− ES cells into both flanks of a cohort of nude mice and tumours were excised at 2, 3, 3.5, 4 and 8 weeks after implantation for whole mount X-gal staining (Table 1 shows the numbers of tumours analysed). As the blood supply to the developing tumour can be contributed through the nude mouse vascular system, we observed no significant difference in tumour growth rate between Lmo2 +/− and Lmo2-tumours. After about two weeks, an intrinsic ES cell-derived vascular system begins to develop and could be distinguished from host (nude mice) vessels because of Lmo2-lacZ fusion protein in endothelial cells of ES cell origin. Most tumour vessels on the surface of early stage tumour (before 3 weeks after implantation) were negative for X-gal staining. Two weeks after implantation, there was no sign of obvious ES cell-derived vascular tree development in either Lmo2 +/− (FIG. 8A) and Lmo2 −/− tumours (FIG. 8B). However, clusters of lacZ-positive cells (blue in FIGS. 8A and 8B), putative angioblasts (precursors of sprouting endothelium) were present in both sets of tumours. Vascular trees, displaying sprouting neo-vasculature, develop from lacZ positive cell clusters in Lmo2 +/− tumour around 3 weeks after implantation (FIG. 8C), but there was no sign of any vascular tree development in Lmo2 −/− tumours (FIG. 8D). Vascular trees which appeared in Lmo2 +/− tumours kept growing until they formed the more mature vascular system in the tumour at 3.5 weeks (FIG. 8E) and 4 weeks (FIG. 8G). However, there was still no ES-derived vascular tree development in Lmo2 −/− tumour at 3.5 weeks (FIG. 8F) or at 4 weeks (FIG. 8H), other than the clusters of lacZ-positive cells seen at 2 weeks (see table 1). The difference in vascular development between Lmo2 +/− and Lmo2 −/− tumours, however, became less obvious at 8 weeks after implantation (data not shown), probably because nude mouse and ES cell-derived vascular systems fused to form chimaeric vessels at this advanced stage. These data show that Lmo2 is not needed for de novo specification of endothelial precursors (angioblasts) from the mesodermal component in tumour growth, as found in mouse embryo vascular formation, nor for the maintenance of the existing vascular system (because there was no sign of selective apoptosis of Lmo2-null endothelial cells). However, the data show Lmo2 is obligatory in the sprouting step of angiogenesis.

The essential nature of Lmo2 in angiogenesis is confirmed in Lmo2−/− mice constructed using a tetracycline-inducible Lmo2 system. Tet-inducible Lmo2/+ mice are crossed with Lmo2+/− mice as above, and pups rescued by tetracycline administration. Tet-inducible Lmo2/− mice are identified, and converted to Lmo2−/− at maturity by withdrawing tetracycline. MLV infection in control Lmo2+ mice leads to the generation of thymomas in 1005 of cases. However, no thymoma generation is observed in Lmo2−/− mice.

Lmo2 was identified as a T cell oncogene activated by specific chromosomal translocations (Rabbitts et al., 1998). The gene is normally expressed in endothelial cells of mouse embryos where it is required for angiogenesis (endothelial cell re-modelling). The current data show Lmo2 expression in adult mouse tissues and augmentation of levels of expression in tumour neo-vasculature. Furthermore, in the absence of Lmo2, the development of a mature vascular system in solid tumours is prevented. Neo-vascularisation is a crucial step in development of many human diseases, such as cancer and diabetic retinopathy so the regulation of this process could control these progressive diseases. Although growth factors and their cell surface receptors which are important for angiogenesis (e.g. vascular endothelial growth factor, angiopoietin, fibroblast growth factor and their cell surface receptors) and their clinical application have been extensively studied, the molecular mechanism of transcriptional control of angiogenesis remains unclear. Our study on the LIM-only protein Lmo2 which is a nuclear regulator of tumour neo-vascularisation highlights an important therapeutic application to cancer treatment, especially because Lmo2 has a specific role in neo-vascularisation whilst existing endothelial cells do not seem to require the protein. By modifying the LIM-domain protein-protein interactions of Lmo2, we might be able to control the critical specific step of neo-vascularisation. Finally by inhibiting the transcriptional mechanism of neo-vascularisation, it should be possible to simultaneously control angiogenesis-related downstream genes, which may be a more potent method to regulate neo-vascularisation and eventually to control tumour growth and metastasis.

TABLE I

| | | | | | |
|---|---|---|---|---|---|
| Embryonic | Ut. Sacs | Embryos | | LacZ + ve | |
| day | (A) | (B) | B/A % | (C) | C/B % |

A. KZ26(+/−) chimaeric mice

| | | | | | |
|---|---|---|---|---|---|
| E9.5 | 30 | 30 | 100 | 15 | 50 |
| E10.5 | 18 | 18 | 100 | 13 | 72 |
| E11.5 | 15 | 15 | 100 | 11 | 73 |
| E12.5 | 15 | 15 | 100 | 10 | 67 |

B. KZ26(+/−) chimaeric mice

| | | | | | |
|---|---|---|---|---|---|
| E9.5 | 12 | 12 | 100 | 10 | 83 |
| E10.5 | 134 | 117 | 87 | 85 | 73 |
| E11.5 | 92 | 29 | 32 | 7 | 24 |
| E12.5 | 71 | 22 | 31 | 5 | 23 |
| E14.5 | * | 20 | | 8 | 40 |

No. of embryos per clone

| Embryonic day | −/− cl. 1 | −/− cl. 16 | −/− cl. 64 |
|---|---|---|---|
| E9.5 | 10 | 2 | nd |
| E10.5 | 38 | 22 | 57 |
| E11.5 | 12 | 17 | nd |
| E12.5 | 16 | 0 | 6 |
| E14.5 | 13 | 7 | nd |

Ut. Sacs = uterine sacs;
nd = not done.
The symbol * denotes uterine sacs uncountable because of degeneration.

Chimaeric mice were produced from the injection into blastocysts of ES cells with (A) a knock-in of lacZ into one allele of Lmo2 (KZ26 +/−) or (B) a knock-in of lacZ into one allele of Lmo2 and a knock-out of the second allele by insertion of hygromycin (KZ26 +−/−).

These blastocysts were implanted into recipients and at the specified embryonic time points, uterine sacs were counted, embryos were dissected and stained for beta-galactosidase activity with Xgal. The data are expressed as % of live embryos per total uterine sacs (A/B) (those embryos with marked degeneration were not included) and % beta-galactosidase-positive embryos (C/B). For the KZ26 −/− ES clones, the number of embryos examined for each clone is given (note: no clone 64 embryos were examined at E9.5, E11.5 or E14.5).

TABLE 2
ES-derived sprouting angiogenesis
during teratocarcinoma development

| | Number of teratocarcinomas | |
|---|---|---|
| | analysed | |
| Weeks after injection | Lmo2 +/− | Lmo2 −/− |
| 2 | 4 (1) | 4 (0) |
| 3 | 5 (5) | 5 (0) |
| 3.5 | 3 (3) | 5 (0) |
| 4 | 7 (7) | 5 (0) |
| Total with sprouting | 16 (84%) | 0 (0%) |
| angiogenesis | | |

ES cell-derived teratocarcinomas were induced by subcutaneous injection of Lmo2 +/− or Lmo2 −/− ES cells into nude mice. In total 19 tumours from each implantation were examined, and the number at the time point is indicated. The number of tumours at each time point is given in brackets and the percentage shown with the final total.

### REFERENCES

Allen, N. D., Cran, D. G., Barton, S. C., Hettle., S., Reik, W. And Surani, M. A. (1988). Transgenes as probes for active chromosomal domains in mouse development. Nature 333: 852-855.
Boelun T, Buluwela L, Williams D, White L and Rabbitts T H, 1988, A cluster of chromosome 11p13 translocations found via distinct D-D and D-J rearrangements of the human T cell receptor 8 chain gene, The EMBO Journal 7: 2011-2017.
Boehm T, Foroni L, Kaneko Y, Perutz M F and Rabbitts T H, 1991, The rhombotin family of cysteine-rich LIM-doman oncogenes: Distinct members are involved in T-cell translocations to human chromosomes 11p15 and 11p13, Proc. Natl. Acad. Sci. USA 88: 4367-4371.
Dear, T. N., Colledge, W. H., Carlton, M. B. L., Lavenir, I., Larson, T., Smith, A. J. H., Warren, A. J., Evans, M. J., Sofroniew, M. V. and Rabbitts, T. H. (1995). The Hoxl1 gene is essential for cell survival during spleen development. Devlopment 212: 2909-2915.
Fisch, P., Boehm, T., Lavenir, I., Larson, T., Arno, J., Forster, A. & Rabbitts, T. H. T-cell acute lymphoblastic lymphoma induced in transgenic mice by the RBTN1 and RBTN2 LIM-domain genes Oncogene 7, 2389-2397 (1992).

Foroni L, Boehm T, White L, Forster A, Sherrington P, Liao X B, Brannan C I, Jenkins A, Copeland N G and Rabbitts T H, 1992, The Rhombotin Gene Family Encode Related LIM-domain Proteins whose Differing Expression Suggests Multiple Roles in Mouse Development, J. Mol. Biol. 226: 747-761.

Hanahan, D. & Folkman, J. Patterns and emerging mechanisms of the angiogenic switch during tumorigenesis. Cell 86, 353-364 (1996).

Larson, R., Fisch, P., Larson, T., Lavenir, I., Langford, T., King, G. & Rabbitts, T. H. T cell tumours with disparate phenotypes develop with long latency in mice transgenic for rbtn2 Oncogene 9, 3675-3681 (1994).
Larson, R. C., Osada, H., Larson, T. A., Lavenir, I. & Rabbitts, T. H. The oncogenic LIM protein Rbtn2 causes thymic developmental aberrations that precede malignancy in transgenic mice Oncogene 11, 853-862 (1995).
Larson R C, Lavenir I, Larson T A, Baer R, Warren A J, Wadman I, Nottage K and Rabbitts T H, 1996, Protein dimerization between Lmo2 (Rbtn2) and Tal1 alters thymocyte development and potentiates T cell tumorigenesis in transgenic mice, The EMBO Journal, Vol 15: 1021-1027.
LeFranc, M.-P., Forster, A., Baer, R., Stinson, M. A. and Rabbitts, T. H. (1986). Diversity and rearrangement of the human T cell rearranging gamma genes: Nine germ-line variable genes belonging to two subgroups. Cell 45: 237-246.
Rabbitts, T. H. LMO T-cell translocation oncogenes typify genes activated by chromosomal translocations that alter transcription and developmental processes Genes & Devel. 12, 2651-2657 (1998).
Valge-Archer V E, Osada H, Warren A J, Forster A, Li J, Baer R and Rabbitts T H, 19094, The LIM protein RBTN2 and the basic helix-loop-helix protein TAL1 are present in a complex in erythroid cells, Proc. Natl. Acad. Sci. USA, Vol 91: 8617-8621.
Valge-Archer V, Forster A and Rabbitts T H, 1998, The LMO1 and LDB1 proteins interact in human T cell acute leukaemia with the chromosomal translocation t(11; 14) (p15;q11), Oncogene (1998), Vol 17: 3199-3202.
Wadman I A, Osada H, Grütz G G, Agulnick A D, Westphal H, Forster A, and Rabbitts T H, 1997, The LIM-only protein Lmo2 is a bridging molecule assembling an erythroid, DNA-binding complex which includes the TAL1, E47, GAT-1 and Ldb1/NLI proteins, The EMBO Journal 16: 3145-3157.
Wadman I, Li J, O. Bash R, Forster A, Osada H, Rabbitts T H and Baer R, 1994, Specifc in vivo association between the bHLH and LIM proteins implicated in human T cell leukemia, The EMBO Journal 13: 4831-4839.
Warren A J, Colledge W H, Carlton M B L, Evans M J, Smith A J H and Rabbits T H, 1994, The Oncogenic Cysteine-Rich LIM Domain Protein Rbtn2 Is Essential for Erythroid Development, Cell 78: 45-57.
Yamada Y, Warren A J, Dobson C, Forster A, Pannell R and Rabbitts T H, 1998, The T cell leukemia LIM protein Lmo2 is necessary for adult mouse haematopoiesis, Proc. Natl. Acad, Sci. USA 95: 3890-3895.

1: A method of modulating angiogenesis in a mammal, comprising administering a substance to the mammal that modulates the expression and/or function of Lmo2 or a functionally related polypeptide, wherein angiogenesis is inhibited.

2: A method according to claim 1, wherein the expression and/or function of the Lmo2 or functionally related polypeptide is inhibited.

3: A method according to claim 1, wherein angiogenesis is inhibited in a specific tissue of said mammal.

4: A method for inhibiting the contribution of a cell to a process of angiogenesis in a mammal, comprising administering a substance that inhibits the expression of Lmo2 in said cell.

5: A method according to claim 1, wherein said substance is identified by a method comprising:
contacting Lmo2 and/or the functionally related polypeptide with a candidate substance, and
determining whether the angiogenesis mediating activity of Lmo2 or the functionally related polypeptide is inhibited.

6: A method according to claim 5, wherein said substance binds to the Lmo2 or the functionally related polypeptide.

7: A method according to claim 5, wherein said substance affects the interaction between the Lmo2 and the functionally related polypeptide.

8: A method according to claim 5, wherein said substance down regulates the expression of the Lmo2 or the functionally related polypeptide.

9: A method according to claim 5, wherein said substance inhibits the function of the Lmo2 or the functionally related polypeptide.

10: A method according to any one of claims 5 or 6-9, wherein the Lmo2 or the functionally related polypeptide is expressed on a cell.

11: A method according to claim 5, wherein the functionally related polypeptide is a Lmo2 binding partner.

12: A method according to claim 6, wherein the functionally related polypeptide is a Lmo2 binding partner.

13: A method according to claim 7, wherein the functionally related polypeptide is a Lmo2 binding partner.

14: A method according to claim 8, wherein the functionally related polypeptide is a Lmo2 binding partner.

15: A method according to claim 9, wherein the functionally related polypeptide is a Lmo2 binding partner.

16: A method according to claim 10, wherein the functionally related polypeptide is a Lmo2 binding partner.
